(19) United States
(12) Patent Application Publication
(10) Pub. No.: US 2026/0330669 A1
(43) Pub. Date: Oct. 22, 2026

(54) METHOD FOR CONSTRUCTING GLIOMA CHEMOTHERAPY RESPONSE PREDICTION MODEL, DEVICE, AND MEDIUM

(71) Applicant: BEIJING INSTITUTE OF NEUROSURGERY, Beijing City (CN)

(72) Inventors: Yinyan WANG, Beijing City (CN); Zihan WANG, Beijing City (CN); Zeya YAN, Beijing City (CN); Hongbo BAO, Beijing City (CN); Jie HU, Beijing City (CN); Renwu ZHANG, Beijing City (CN); Ruiyang WANG, Beijing City (CN); Tao JIANG, Beijing City (CN)

(73) Assignee: BEIJING INSTITUTE OF NEUROSURGERY, Beijing City (CN)

(21) Appl. No.: 19/648,749

(22) Filed: Apr. 15, 2026

(30) Foreign Application Priority Data
Apr. 16, 2025 (CN) 202510472700.9

(51) Int. Cl. G06T 7/62; A61B 5/00; A61B 5/055; G06T 7/00; G06V 10/776; G16H 50/20

(52) U.S. Cl. CPC G06T 7/62; A61B 5/0036; A61B 5/055; G06T 7/0016; G06V 10/776; G16H 50/20; G06T 2207/10016; G06T 2207/10088; G06T 2207/20081; G06T 2207/30096

(57) ABSTRACT

A method for constructing a glioma chemotherapy response prediction model, a device, and a medium are provided. In the present disclosure, the features of magnetic resonance images based on the radiomic technology are extracted and screened, thereby constructing a sample dataset, different machine learning models are trained by using the sample dataset, and then an optimal trained machine learning model is selected as the glioma chemotherapy response prediction model.

### CROSS REFERENCE TO RELATED APPLICATION

This patent application claims the benefit and priority of Chinese Patent Application No. 202510472700.9 filed with the China National Intellectual Property Administration on Apr. 16, 2025, the disclosure of which is incorporated by reference herein in its entirety as part of the application.

### TECHNICAL FIELD

The present disclosure relates to the technical field of medical devices, and in particular, to a method for constructing a glioma chemotherapy response prediction model, a device, and a medium.

### BACKGROUND

Due to the infiltrative nature of glioma, it is difficult to completely remove tumor cells from brain tissue through surgical intervention alone. Consequently, patients with gliomas typically require adjuvant chemotherapy following neurosurgery. However, the response to chemotherapy varies widely due to tumor heterogeneity. Patients who are sensitive to chemotherapy can achieve median overall survival greater than 20 months, while non-responders often survive less than 12 months. These disparities in treatment response not only influence survival but also expose non-responders to unnecessary toxicity, such as neurocognitive decline, gastrointestinal complications, and hematologic toxicity, along with significant financial burden. Nevertheless, in current clinical practice, there is a lack of reliable methods to identify, prior to treatment initiation, those patients who are likely to respond favorably to chemotherapy. As a result, most patients are administered standardized chemotherapy regimens in a non-selective manner. Accordingly, there exists an urgent clinical and technical need for methods capable of predicting a patient's response to chemotherapy before treatment, so as to enable individualized therapeutic decision-making and to reduce unnecessary physical, psychological, and financial burdens associated with ineffective treatment.

### SUMMARY

An objective of the present disclosure is to provide a method for constructing a glioma chemotherapy response prediction model, a device, and a medium, thereby establishing a glioma chemotherapy response prediction model and achieving the prediction of the glioma chemotherapy response.

In order to achieve the above objective, the present disclosure provides the following solutions.

In a first aspect, the present disclosure provides a method for constructing a glioma chemotherapy response prediction model, which includes the following steps.

A longitudinal multi-time-point magnetic resonance imaging dataset is acquired. The longitudinal multi-time-point magnetic resonance imaging dataset includes a plurality of magnetic resonance imaging sequences. The magnetic resonance image sequence includes magnetic resonance image groups of high-grade gliomas from the same patient at least at three time points; the three time points are located before surgery, before adjuvant chemotherapy, and after completion of adjuvant chemotherapy, respectively; and the magnetic resonance image group includes one or more of a T1-weighted image, a T2-weighted image, and a contrast-enhanced T1-weighted image.

A volume change of residual tumors in the magnetic resonance image group before adjuvant chemotherapy and in the magnetic resonance image group after completion of adjuvant chemotherapy in each of the magnetic resonance imaging sequences is calculated as a volume change corresponding to each of the magnetic resonance imaging sequences.

Chemotherapy sensitivity corresponding to each of the magnetic resonance imaging sequences is determined according to the volume change corresponding to each of the magnetic resonance imaging sequences.

Features of the magnetic resonance image group before surgery in each of the magnetic resonance imaging sequences are extracted and screened to obtain a feature set corresponding to each of the magnetic resonance imaging sequences.

The feature set corresponding to each of the magnetic resonance imaging sequences is used as input and the chemotherapy sensitivity corresponding to each of the magnetic resonance imaging sequences is used as a label, to construct a sample dataset.

A plurality of machine learning models are trained based on the sample dataset, and an optimal trained machine learning model is selected as the glioma chemotherapy response prediction model.

In a second aspect, the present disclosure provides a computer device, including a memory, a processor, and a computer program stored in the memory and executable on the processor. The processor is configured to execute the computer program to implement the method for constructing the glioma chemotherapy response prediction model described above.

In a third aspect, the present disclosure provides a computer-readable storage medium, having a computer program stored thereon. The computer program, when executed by a processor, implements the method for constructing the glioma chemotherapy response prediction model described above.

According to the specific embodiments provided by the present disclosure, the present disclosure has the following technical effects.

The present disclosure provides a method for constructing a glioma chemotherapy response prediction model, a device, and a medium. In the present disclosure, the features of magnetic resonance images are extracted and screened based on the radiomic technology to construct a sample dataset, different machine learning models are trained by using this sample dataset, and then the optimal trained machine learning model is selected as the glioma chemotherapy response prediction model, which achieves the prediction of the glioma chemotherapy response.

### BRIEF DESCRIPTION OF THE DRAWINGS

In order to more clearly explain the technical solutions in the embodiments of the present disclosure or the conventional art, the drawings needed to be used in the embodiments will be briefly introduced below. Obviously, the drawings described below are only some embodiments of the present disclosure. For those skilled in the art, other drawings may be obtained according to these drawings without paying creative labor.

FIG. 1 is a flow chart of a method for constructing a glioma chemotherapy response prediction model according to an embodiment of the present disclosure.

FIG. 2 is a graph of a receiver operating characteristic curve of a glioma chemotherapy sensitivity prediction model according to an embodiment of the present disclosure.

FIG. 3 is a structural diagram of a computer device according to an embodiment of the present disclosure.

### DETAILED DESCRIPTION OF THE EMBODIMENTS

The technical solutions in the embodiments of the present disclosure will be clearly and completely described with reference to the drawings in the embodiments of the present disclosure hereinafter. Obviously, the described embodiments are only some of the embodiments of the present disclosure, rather than all of the embodiments. Based on the embodiments in the present disclosure, all other embodiments obtained by those skilled in the art without paying creative labor belong to the scope of protection of the present disclosure.

To make the above objective, features and advantages of the present disclosure more obvious and understandable, the present disclosure will be further described in detail with reference to the drawings and the specific embodiments.

Radiomics is a product of the development and advancement of image pattern recognition technologies, which can extract a large number of objective quantitative features from medical images that are difficult to discern with naked eyes, including signal strength distribution, three-dimensional shapes, texture features, and wavelet-transformed features of tumor images. This technology allows for a quantitative characterization of solid tumor image features, and offers a new approach for predicting and analyzing clinically relevant events in the diagnosis and treatment of such a disease. In the field of glioma, radiomics has demonstrated broad application potential across multiple domains, especially in therapeutic response prediction. For example, a radiomic model developed by Grossmann P, Kickingereder P et al. can effectively screen patients with recurrent glioblastoma who may benefit from bevacizumab (anti-angiogenic therapy). Similarly, George E et al. constructed another radiomic model to identify patients with recurrent glioblastoma who may benefit from durvalumab (immunotherapy). These models provide an important support for individualized treatment and are expected to improve the therapeutic effect of patients.

Magnetic Resonance Imaging (MRI) plays an important role in diagnosis, treatment planning and follow-up monitoring of gliomas. Different MRI sequences, including a T1-weighted image (T1 for short), a T2-weighted image (T2 for short), a contrast-enhanced T1-weighted image (CE for short), and Fluid Attenuated Inversion Recovery (FLAIR), provide complementary information for assessing the image features such as structures, edema and necrosis of tumors. In clinical practice, the therapeutic response of gliomas is usually assessed according to Response Assessment in Neuro-Oncology (RANO), which assesses the therapeutic effect by monitoring the dynamic change of a tumor volume based on longitudinal multi-time-point MRI scanning. In view of the core role of MRI in glioma management, the radiomic model based on MRI data has become an ideal tool to predict chemotherapy sensitivity of gliomas, which is helpful to achieve an individualized treatment plan.

The MRI data is used to construct a glioma chemotherapy response prediction model to predict the therapeutic response of a high-grade glioma patient to chemotherapy, which is expected to assist physicians in selecting the most appropriate personalized treatment strategies, improving the treatment outcomes and reducing unnecessary physical, psychological, and financial burden.

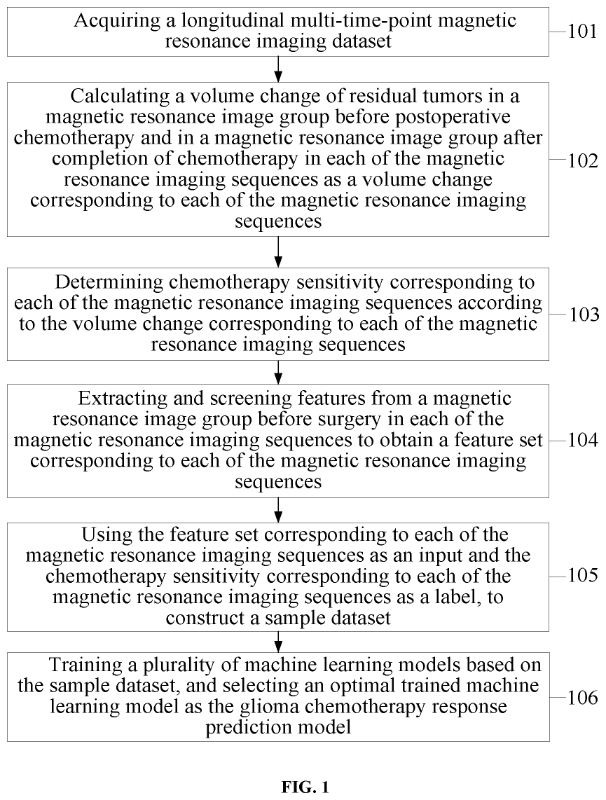

In an exemplary embodiment, as shown in FIG. 1, a method for constructing a glioma chemotherapy response prediction model is provided, including step 101 to step 106.

In step 101, a longitudinal multi-time-point magnetic resonance imaging dataset is acquired. The longitudinal multi-time-point magnetic resonance imaging dataset includes a plurality of magnetic resonance imaging sequences. The magnetic resonance image sequence includes magnetic resonance image groups of high-grade gliomas from the same patient at least at three time points; the three time points are located before surgery (i.e., preoperative), before adjuvant chemotherapy (i.e., after surgery and before chemotherapy), and after completion of adjuvant chemotherapy (i.e., post-chemotherapy), respectively; and the magnetic resonance image group includes one or more of a T1-weighted image, a T2-weighted image, and a contrast-enhanced T1-weighted image.

In step 102, a volume change of residual tumors in the magnetic resonance image group before adjuvant chemotherapy and in the magnetic resonance image group after completion of adjuvant chemotherapy in each of the magnetic resonance imaging sequences is calculated as the volume change corresponding to each of the magnetic resonance imaging sequences.

In step 103, chemotherapy sensitivity corresponding to each of the magnetic resonance imaging sequences is determined according to the volume change corresponding to each of the magnetic resonance imaging sequences.

In step 104, features of the magnetic resonance image group before surgery in each of the magnetic resonance imaging sequences are extracted and screened to obtain a feature set corresponding to each of the magnetic resonance imaging sequences.

In step 105, the feature set corresponding to each of the magnetic resonance imaging sequences is used as input and the chemotherapy sensitivity corresponding to each of the magnetic resonance imaging sequences is used as a label, to construct a sample dataset.

In step 106, a plurality of machine learning models are trained based on the sample dataset, and an optimal trained machine learning model is selected as the glioma chemotherapy response prediction model.

By implementing the above step 101 to step 106, a glioma chemotherapy response prediction model may be established to achieve the prediction of the chemotherapy response.

In another exemplary embodiment of the present disclosure, the above step 101 is implemented in the following manner.

The longitudinal multi-time-point magnetic resonance imaging dataset of high-grade gliomas (Word Health Organization, WHO grades 3 to 4) is collected from a glioma database. The longitudinal multi-time-point magnetic resonance imaging dataset includes a plurality of magnetic resonance imaging sequences. The magnetic resonance image sequence includes magnetic resonance image groups of high-grade gliomas from the same patient at least at three time points. The magnetic resonance image group includes a T1-weighted image, a T2-weighted image, and a contrast-enhanced T1-weighted image. The specific time points collected are the time points before surgery, within one week before adjuvant chemotherapy and four months after postoperative chemotherapy.

The magnetic resonance imaging (MRI) technology can highlight different types of tissues or pathological features through different scanning sequences and parameter settings. The T1-weighted image, the T2-weighted image, and the contrast-enhanced T1-weighted image are common imaging sequences, which are of great significance in different clinical applications.

The T1-weighted image is an image obtained by setting shorter Repetition Time (TR) and Echo Time (TE). T1 represents “Spin-lattice relaxation time”, which is, the time required for water molecules to return to equilibrium through the interaction between water molecules and surrounding tissues. The T1-weighted image is usually used to visualize an anatomical structure.

The T2-weighted image is an image obtained through longer repetition time and echo time. T2 represents “Spin-spin relaxation time”, that is, the time when energy disappears due to the interaction between water molecules and the surrounding environment. The T2-weighted image is suitable for showing cerebral edema, tumors, inflammation and other pathological changes.

The contrast-enhanced T1-weighted image uses a contrast agent (usually a gadolinium-containing contrast agent) to enhance the signal of the image based on T1-weighted imaging, thereby helping improve the visibility of some lesion areas. The contrast agent is introduced into the bloodstream through intravenous injection, and is accumulated at specific lesion sites, thereby increasing signal intensity at these sites. The contrast-enhanced T1-weighted image is widely used to detect, locate and assess the blood supply of tumors, and can also aid in identifying some other types of lesions, such as infection and spinal cord abnormalities.

In another exemplary embodiment of the present disclosure, step 102 of the present disclosure calculates the volume change of residual tumors before and after completion of adjuvant chemotherapy based on the contrast-enhanced T1-weighted image before and after completion of adjuvant chemotherapy collected in step 101, specifically including step 201 to step 204.

In step 201, automatic tumor area annotation is performed on the contrast-enhanced T1-weighted image before and after completion of adjuvant chemotherapy by using the deep learning model.

In step 202, information about the number of voxels contained in a residual tumor area and the volume of a single voxel is extracted.

In step 203, the volumes of residual tumors before and after completion of adjuvant chemotherapy are calculated according to the formulas V₁=N₁×V₀ and V₂=N₂×V₀, where V₁ is the volume of residual tumor in the magnetic resonance image group before adjuvant chemotherapy, that is, the first volume, N₁ is the number of voxels contained in a residual tumor area in the magnetic resonance image group before adjuvant chemotherapy, V₀ is the volume of a single voxel, V₂ is the volume of residual tumor in the magnetic resonance image group after completion of adjuvant chemotherapy, that is, the second volume, and N₂ is the number of voxels contained in a residual tumor area in the magnetic resonance image group after completion of adjuvant chemotherapy.

In step 204, a volume change is calculated according to the formula

$$Δ V = \frac{{V}_{1} - {V}_{2}}{{V}_{1}} \times 100 \% ,$$

where ΔV is the volume change.

In another exemplary embodiment of the present disclosure, in step 103, the volume change of tumors before and after completion of adjuvant chemotherapy is compared according to the response assessment in neuro-oncology to determine the chemotherapy sensitivity, and the specific assessment criteria is as follows.

If the tumor disappears completely after treatment, it is classified as a complete response; if the tumor volume decreases by ≥65% following treatment, it is defined as a partial response; if the tumor volume decreases by <65% but increases by <40%, it is defined as stable disease; and if the tumor volume increases by ≥40%, it is defined as progressive disease. The complete response and the partial response are determined as chemosensitive, while those with stable disease and progressive disease are regarded as chemoresistant.

In another exemplary embodiment of the present disclosure, in step 104 above, radiomic features of the magnetic resonance image group before surgery are extracted, including a T1-weighted image, a T2-weighted image, and a contrast-enhanced T1-weighted image. Then, the obtained radiomic features are screened to screen out differential radiomic features of patients with different therapeutic responses.

The obtained radiomic features are screened to screen out differential radiomic features of patients with different therapeutic responses, specifically including as the following. First, for the obtained radiomic features, a feature subset with the strongest distinctiveness is identified by using a Maximum Relevancy Minimum Redundancy (mRMR) algorithm, and the top 50 features are retained. Then, Spearman correlation analysis is performed to assess the inter-feature correlations among these top-ranked features. For the features with high correlation (r>0.9 and p<0.05, where r is a correlation coefficient of a Spearman's rank correlation test, and p is a statistical significance level), only the feature with a larger variance providing greater discriminative information is retained. Finally, by using a Least Absolute Shrinkage and Selection Operator (LASSO) algorithm, and combined with ten-fold cross-validation, the most predictive radiomic features are further screened out from the remaining features to form a feature set. For example, the radiomic features selected in this step include the following 12 features:
1. original_ngtdm_Strength_T2−T1: strength of an original neighborhood gray-tone difference matrix_T2−T1;
2. log-sigma-5-0-mm-3D_glszm_ZoneVariance_T1: a zone variance of a log-σ-5-0-mm-3D gray-level size zone matrix_T1;
3. log-sigma-5-0-mm-3D_glszm_ZonePercentage_T1: a zone percentage of the log-σ-5-0-mm-3D gray-level size zone matrix_T1;
4. wavelet-LLH_glcm_JointAverage_CE: a joint average of a wavelet-LLH gray-level co-occurrence matrix_CE;
5. log-sigma-5-0-mm-3D_ngtdm_Contrast_T2: a contrast of a log-σ-5-0-mm-3D neighborhood gray-tone difference matrix_T2;
6. wavelet-LLH_glszm_GrayLevelVariance_T2: a gray-level variance of a wavelet-LLH gray-level size zone matrix_T2;
7. wavelet-LLH_glrlm_RunVariance_T2: a run-length variance of a wavelet-LLH gray-level run-length matrix_T2;
8. wavelet-LLH_glrlm_ShortRunEmphasis_CE: a short run-length emphasis of the wavelet-LLH gray-level run-length matrix_CE;
9. wavelet-LLH_glrlm_RunLengthNonUniformityNormalized_T2: run-length non-uniformity normalized of the wavelet-LLH gray-level run-length matrix_T2;
10. wavelet-LLH_glrlm_RunLengthNonUniformity_T2: run-length non-uniformity of the wavelet-LLH gray-level run-length matrix_T2;
11. wavelet-LLH_glrlm_RunEntropy_T2: a run-length entropy of the wavelet-LLH gray-level run-length matrix_T2; and
12. wavelet-LLH_glrlm_LongRunLowGrayLevelEmphasis_T2: long-run-length low-gray-level emphasis of the wavelet-LLH gray-level run-length matrix_T2.

The suffixes T2, T1 and CE indicate that the features are extracted from the T2-weighted image, the T1-weighted image, and the contrast-enhanced T1-weighted image, respectively; and the suffix T2−T1 indicates that the feature is the difference between the feature values extracted from the T2-weighted image and the T1-weighted image.

In an exemplary embodiment, in step 106 above, the machine learning models are trained by using the screened radiomic features and their labels to form a glioma chemotherapy response prediction model, specifically including step 301 to step 303.

In step 301, first, the sample dataset is divided into a training set and a test set according to a ratio of 8:2.

In step 302, a random forest model, a support vector machine model, an extreme gradient boosting model, and a linear regression model are trained based on the training set by using the machine learning algorithms, to train corresponding predictive models.

In step 303, the performance of each model is assessed based on the test set, the area under the receiver operating characteristic curve, sensitivity, specificity, and accuracy are calculated, and the model with an optimal performance is selected as the glioma chemotherapy response prediction model.

For example, after comparison, the linear regression model is optimal, and the final obtained glioma chemotherapy response prediction model is shown in the following formula:

R=0.5046+0.0203×M₁+0.0645×M₂+0.0063×M₃+0.0614×M₄+0.0017×M₅−0.0015×M₆−0.0619×M₇−0.0704×M₈−0.0459×M₉−0.0000×M₁₀+0.0284×M₁₁−0.0403×M₁₂

where R is a score of chemotherapy response sensitivity, when R>0.5, a predicted result is determined as chemosensitive, otherwise, the predicted result is determined as chemoresistant, and M₁, M₂, M₃, M₄, M₅, M₆, M₇, M₈, M₉, M₁₀, M₁₁, and M₁₂ denote strength of an original neighborhood gray-tone difference matrix, a zone variance of a log-σ-5-0-mm-3D gray-level size zone matrix, a zone percentage of the log-σ-5-0-mm-3D gray-level size zone matrix, a joint average of a wavelet-LLH gray-level co-occurrence matrix, a contrast of a log-σ-5-0-mm-3D neighborhood gray-tone difference matrix, a gray-level variance of a wavelet-LLH gray-level size zone matrix, a run-length variance of a wavelet-LLH gray-level run-length matrix, a short run-length emphasis of the wavelet-LLH gray-level run-length matrix, run-length non-uniformity normalized of the wavelet-LLH gray-level run-length matrix, run-length non-uniformity of the wavelet-LLH gray-level run-length matrix, a run-length entropy of the wavelet-LLH gray-level run-length matrix, and long-run-length low-gray-level emphasis of the wavelet-LLH gray-level run-length matrix, respectively.

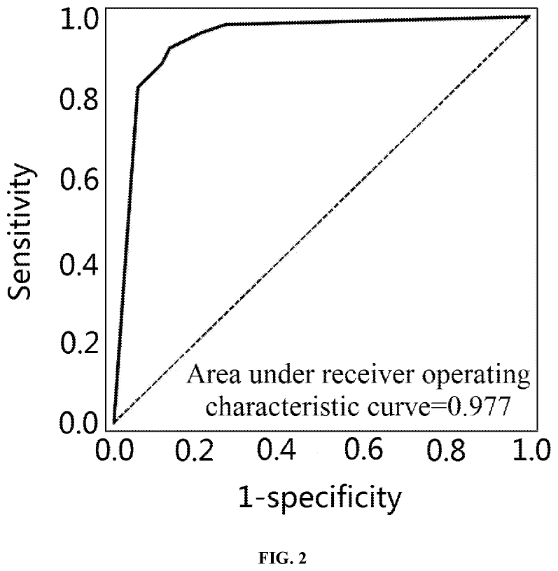

As shown in FIG. 2, a receiver operating characteristic curve of a glioma chemotherapy sensitivity prediction model is shown in FIG. 2. The X-axis is “1-specificity”, which indicates the proportion of negative samples which are incorrectly classified as positive samples by the model; and the Y-axis is “sensitivity”, which indicates the proportion of positive samples correctly identified by the model. The area under the receiver operating characteristic curve of the glioma chemotherapy sensitivity prediction model is 0.977, demonstrating excellent overall performance and suitability for high-precision classification tasks.

According to the specific embodiments provided by the present disclosure, the present disclosure has the following technical effects.

In the embodiment of the present disclosure, the high-throughput features are extracted from preoperative magnetic resonance images of patients with gliomas. Based on these features, the most critical features for predicting postoperative chemotherapy response are identified. Subsequently, these key features are utilized to train various machine learning models to predict the postoperative therapeutic response of patients to chemotherapy.

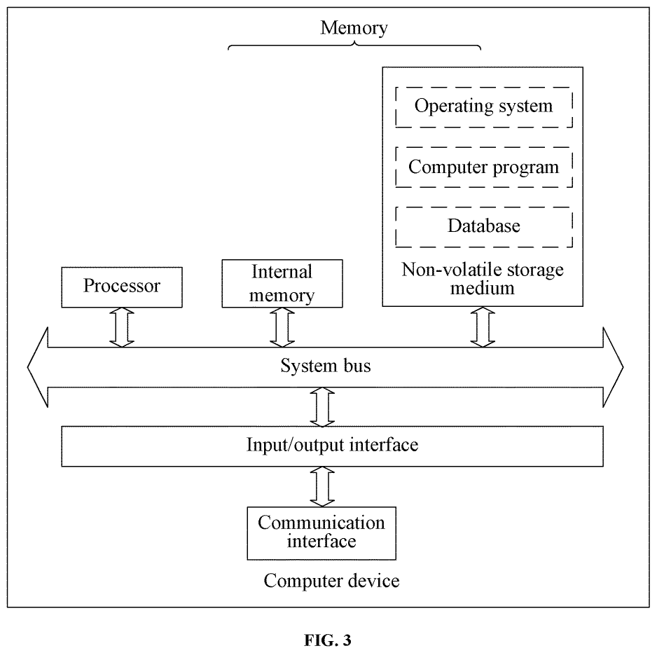

In an exemplary embodiment, a computer device is provided. The computer device may be a server or a terminal, and the internal structure diagram may be as shown in FIG. 3. The computer device includes a processor, a memory, an Input/Output (I/O) interface and a communication interface. The processor, the memory and the input/output interface are connected through the system bus, and the communication interface is connected to the system bus through the input/output interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for the operation of the operating system and the computer program in the non-volatile storage medium. The input/output interface of the computer device is configured to exchange information between the processor and the external device. The communication interface of the computer device is configured to communicate with an external terminal through network connection. The computer program, when executed by a processor, implements a method for constructing a glioma chemotherapy response prediction model.

It can be understood by those skilled in the art that the structure shown in FIG. 3 is only a block diagram of a part of the structure related to the solution of the present disclosure, and does not constitute a limitation on the computer device to which the solution of the present disclosure is applied. The specific computer device may include more or less components than those shown in the figure, or combine some components, or have different component arrangements.

In an exemplary embodiment, a computer device is provided, including a memory and a processor. A computer program is stored in the memory. The processor, when executing the computer program, implements the steps in the above method embodiments.

In an exemplary embodiment, a computer-readable storage medium is provided, having a computer program stored thereon. The computer program, when executed by a processor, implements the steps in the above method embodiments.

It should be noted that the user information (including but not limited to user equipment information, user personal information, and the like) and data (including but not limited to data for analysis, stored data, displayed data, and the like) involved in the present disclosure are all information and data authorized by users or fully authorized by all parties.

Those skilled in the art can understand that all or part of the processes in the method of the above-mentioned embodiments can be completed by instructing relevant hardware through a computer program. The computer program can be stored in a non-volatile computer-readable storage medium. The computer program, when executed, can include the processes of the embodiments of each of the above-mentioned methods. Any reference to the memory, the database or other media used in each of the embodiments provided by the present disclosure may include at least one of a non-volatile memory and a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto-Resistive Random Access Memory (MRAM), a Ferroelectric Random Access Memory (FRAM), a Phase Change Memory (PCM), a graphene memory, and the like. The volatile memory may include a Random Access Memory (RAM) or an external cache memory. By way of illustration and not limitation, the RAM can be in various forms, such as a Static Random Access Memory (SRAM) or a Dynamic Random Access Memory (DRAM).

The database involved in various embodiments provided by the present disclosure may include at least one of a relational database and a non-relational database. The non-relational database may include, but is not limited to, a distributed database based on a block chain. The processor involved in each of the embodiments provided by the present disclosure may include, but is not limited to, a general processor, a central processing unit, a graphics processing unit, a digital signal processor, a programmable logic device, a data processing logic device based on quantum computing, and the like.

Each of the technical features of the above embodiments can be combined at will. In order to make the description concise, not all possible combinations of each of the technical features in the above embodiments are described. However, as long as there is no contradiction between the combinations of these technical features, the combinations should be considered as the scope recorded in this specification.

In the present disclosure, specific examples are used to explain the principle and implementation of the present disclosure. The description of the above embodiments is only used to help understand the method and the core idea of the present disclosure. At the same time, for those skilled in the art, according to the idea of the present disclosure, there will be changes in the specific implementation and the application scope. To sum up, the contents of this specification should not be construed as limiting the present disclosure.

1. A method for constructing a glioma chemotherapy response prediction model, comprising:
acquiring a longitudinal multi-time-point magnetic resonance imaging dataset, wherein the longitudinal multi-time-point magnetic resonance imaging dataset comprises a plurality of magnetic resonance imaging sequences, each of the plurality of magnetic resonance imaging sequences comprises magnetic resonance image groups of high-grade gliomas from a same patient at least at three time points; the three time points are located before surgery, before adjuvant chemotherapy, and after completion of adjuvant chemotherapy, respectively; and each of the magnetic resonance image groups comprises one or more of a T1-weighted image, a T2-weighted image, and a contrast-enhanced T1-weighted image;
calculating a volume change of residual tumors in a magnetic resonance image group before adjuvant chemotherapy and in a magnetic resonance image group after completion of adjuvant chemotherapy in each of the plurality of magnetic resonance imaging sequences as a volume change corresponding to each of the plurality of magnetic resonance imaging sequences;
determining chemotherapy sensitivity corresponding to each of the plurality of magnetic resonance imaging sequences according to the volume change corresponding to each of the plurality of magnetic resonance imaging sequences;
extracting and screening features from a magnetic resonance image group before surgery in each of the plurality of magnetic resonance imaging sequences to obtain a feature set corresponding to each of the plurality of magnetic resonance imaging sequences;
using the feature set corresponding to each of the plurality of magnetic resonance imaging sequences as an input and the chemotherapy sensitivity corresponding to each of the plurality of magnetic resonance imaging sequences as a label, to construct a sample dataset; and
training a plurality of machine learning models based on the sample dataset, and selecting an optimal trained machine learning model as the glioma chemotherapy response prediction model.

2. The method for constructing the glioma chemotherapy response prediction model according to claim 1, wherein the calculating a volume change of residual tumors in a magnetic resonance image group before adjuvant chemotherapy and in a magnetic resonance image group after completion of adjuvant chemotherapy in each of the plurality of magnetic resonance imaging sequences comprises:
calculating a volume of a residual tumor in the magnetic resonance image group before adjuvant chemotherapy as a first volume by using the following formula:
V₁=N₁×V₀;
where V₁ is the volume of the residual tumor in the magnetic resonance image group before adjuvant chemotherapy, which is the first volume, N₁ is a number of voxels contained in a residual tumor area in the magnetic resonance image group before adjuvant chemotherapy, and V₀ is a volume of a single voxel;
calculating a volume of a residual tumor in the magnetic resonance image group after adjuvant chemotherapy as a second volume by using the following formula:
V₂=N₂×V₀;
where V₂ is the volume of the residual tumor in the magnetic resonance image group after completion of adjuvant chemotherapy, which is the second volume, and N₂ is a number of voxels contained in a residual tumor area in the magnetic resonance image group after completion of adjuvant chemotherapy; and
calculating the volume change according to the first volume and the second volume by using the following formula:
$$Δ V = \frac{{V}_{1} - {V}_{2}}{{V}_{1}} \times 100 \% ;$$
where “ΔV” is the volume change.

3. The method for constructing the glioma chemotherapy response prediction model according to claim 2, wherein the determining chemotherapy sensitivity corresponding to each of the plurality of magnetic resonance imaging sequences according to the volume change corresponding to each of the plurality of magnetic resonance imaging sequences comprises:
determining the chemotherapy sensitivity as chemosensitive in response to determining that “ΔV” is greater than or equal to “65%”, and determining the chemotherapy sensitivity as chemoresistant in response to determining that “ΔV” is less than “65%”.

4. The method for constructing the glioma chemotherapy response prediction model according to claim 2, wherein the extracting and screening features from a magnetic resonance image group before surgery in each of the plurality of magnetic resonance imaging sequences to obtain a feature set corresponding to each of the plurality of magnetic resonance imaging sequences comprises:
extracting features of the magnetic resonance image group before surgery in the magnetic resonance image sequence, to obtain radiomic features of the magnetic resonance image group before surgery in the magnetic resonance image sequence;
calculating distinctiveness of each radiomic feature by using a Maximum Relevancy Minimum Redundancy (mRMR) algorithm, and ranking each radiomic feature in a descending order of distinctiveness to obtain a radiomic feature sequence;
performing correlation analysis on a top predetermined number of radiomic features in the radiomic feature sequence, and selecting radiomic features meeting a predetermined condition from the top predetermined number of radiomic features in the radiomic feature sequence to form a radiomic feature candidate set, wherein the predetermined condition is r>0.9 and p<0.05, wherein r is a correlation coefficient of a Spearman's rank correlation test, and p is a statistical test value; and
selecting a second predetermined number of radiomic features from the radiomic feature candidate set by using a Least Absolute Shrinkage and Selection Operator (LASSO) algorithm, to form a feature set.

5. The method for constructing the glioma chemotherapy response prediction model according to claim 1, wherein the training a plurality of machine learning models based on the sample dataset, and selecting an optimal trained machine learning model as the glioma chemotherapy response prediction model comprises:
dividing the sample dataset into a training set and a test set;
training the plurality of machine learning models by using the training set, to obtain a plurality of trained machine learning models;
testing the plurality of trained machine learning models by using the test set to obtain a test result of each of the plurality of trained machine learning models, wherein the test result comprises one or more of an Area Under Curve (AUC), sensitivity, specificity, and accuracy; and
selecting one of the plurality of trained machine learning models that has an optimal test result as the glioma chemotherapy response prediction model.

6. The method for constructing the glioma chemotherapy response prediction model according to claim 5, wherein the plurality of machine learning models comprise a random forest model, a support vector machine model, an extreme gradient boosting model, and a linear regression model.

7. The method for constructing the glioma chemotherapy response prediction model according to claim 1, wherein the plurality of machine learning models comprise a random forest model, a support vector machine model, an extreme gradient boosting model, and a linear regression model.

8. The method for constructing the glioma chemotherapy response prediction model according to claim 1, wherein the glioma chemotherapy response prediction model is:
R=0.5046+0.0203×M₁+0.0645×M₂+0.0063×M₃+0.0614×M₄+0.0017×M₅−0.0015×M₆−0.0619×M₇−0.0704×M₈−0.0459×M₉−0.0000×M₁₀+0.0284×M₁₁−0.0403×M₁₂;
where “R” is a score of chemotherapy response sensitivity, when “R>0.5”, a predicted result is determined as chemosensitive, and when “R≤0.5”, the predicted result is determined as chemoresistant; and M₁, M₂, M₃, M₄, M₅, M₆, M₇, M₈, M₉, M₁₀, M₁₁, and M₁₂ denote strength of an original neighborhood gray-tone difference matrix, a zone variance of a log-σ-5-0-mm-3D gray-level size zone matrix, a zone percentage of the log-σ-5-0-mm-3D gray-level size zone matrix, a joint average of a wavelet-LowLowHigh (LLH) gray-level co-occurrence matrix, a contrast of a log-σ-5-0-mm-3D neighborhood gray-tone difference matrix, a gray-level variance of a wavelet-LLH gray-level size zone matrix, a run-length variance of a wavelet-LLH gray-level run-length matrix, a short run-length emphasis of the wavelet-LLH gray-level run-length matrix, run-length non-uniformity normalized of the wavelet-LLH gray-level run-length matrix, run-length non-uniformity of the wavelet-LLH gray-level run-length matrix, a run-length entropy of the wavelet-LLH gray-level run-length matrix, and long-run-length low-gray-level emphasis of the wavelet-LLH gray-level run-length matrix, respectively.

9. A computer device, comprising:
a memory,
a processor, and
a computer program, stored in the memory and executable on the processor, wherein the processor is configured to execute the computer program to implement a method for constructing a glioma chemotherapy response prediction model, wherein the method comprises:
acquiring a longitudinal multi-time-point magnetic resonance imaging dataset, wherein the longitudinal multi-time-point magnetic resonance imaging dataset comprises a plurality of magnetic resonance imaging sequences, each of the plurality of magnetic resonance imaging sequences comprises magnetic resonance image groups of high-grade gliomas from a same patient at least at three time points; the three time points are located before surgery, before adjuvant chemotherapy, and after completion of adjuvant chemotherapy, respectively; and each of the magnetic resonance image groups comprises one or more of a T1-weighted image, a T2-weighted image, and a contrast-enhanced T1-weighted image;
calculating a volume change of residual tumors in a magnetic resonance image group before adjuvant chemotherapy and in a magnetic resonance image group after completion of adjuvant chemotherapy in each of the plurality of magnetic resonance imaging sequences as a volume change corresponding to each of the plurality of magnetic resonance imaging sequences;
determining chemotherapy sensitivity corresponding to each of the plurality of magnetic resonance imaging sequences according to the volume change corresponding to each of the plurality of magnetic resonance imaging sequences;
extracting and screening features from a magnetic resonance image group before surgery in each of the plurality of magnetic resonance imaging sequences to obtain a feature set corresponding to each of the plurality of magnetic resonance imaging sequences;
using the feature set corresponding to each of the plurality of magnetic resonance imaging sequences as an input and the chemotherapy sensitivity corresponding to each of the plurality of magnetic resonance imaging sequences as a label, to construct a sample dataset; and
training a plurality of machine learning models based on the sample dataset, and selecting an optimal trained machine learning model as the glioma chemotherapy response prediction model.

10. The computer device according to claim 9, wherein the calculating a volume change of residual tumors in a magnetic resonance image group before adjuvant chemotherapy and in a magnetic resonance image group after completion of adjuvant chemotherapy in each of the plurality of magnetic resonance imaging sequences comprises:
calculating a volume of a residual tumor in the magnetic resonance image group before adjuvant chemotherapy as a first volume by using the following formula:
V₁=N₁×V₀;
where V₁ is the volume of the residual tumor in the magnetic resonance image group before adjuvant chemotherapy, which is the first volume, N₁ is a number of voxels contained in a residual tumor area in the magnetic resonance image group before adjuvant chemotherapy, and V₀ is a volume of a single voxel;
calculating a volume of a residual tumor in the magnetic resonance image group after adjuvant chemotherapy as a second volume by using the following formula:
V₂=N₂×V₀;
where V₂ is the volume of the residual tumor in the magnetic resonance image group after completion of adjuvant chemotherapy, which is the second volume, and N₂ is a number of voxels contained in a residual tumor area in the magnetic resonance image group after completion of adjuvant chemotherapy; and
calculating the volume change according to the first volume and the second volume by using the following formula:
$$Δ V = \frac{{V}_{1} - {V}_{2}}{{V}_{1}} \times 100 \% ;$$
where “ΔV” is the volume change.

11. The computer device according to claim 10, wherein the determining chemotherapy sensitivity corresponding to each of the plurality of magnetic resonance imaging sequences according to the volume change corresponding to each of the plurality of magnetic resonance imaging sequences comprises:
determining the chemotherapy sensitivity as chemosensitive in response to determining that “ΔV” is greater than or equal to “65%”, and determining the chemotherapy sensitivity as chemoresistant in response to determining that “ΔV” is less than “65%”.

12. The computer device according to claim 10, wherein the extracting and screening features from a magnetic resonance image group before surgery in each of the plurality of magnetic resonance imaging sequences to obtain a feature set corresponding to each of the plurality of magnetic resonance imaging sequences comprises:
extracting features of the magnetic resonance image group before surgery in the magnetic resonance image sequence, to obtain radiomic features of the magnetic resonance image group before surgery in the magnetic resonance image sequence;
calculating distinctiveness of each radiomic feature by using a Maximum Relevancy Minimum Redundancy (mRMR) algorithm, and ranking each radiomic feature in a descending order of distinctiveness to obtain a radiomic feature sequence;
performing correlation analysis on a top predetermined number of radiomic features in the radiomic feature sequence, and selecting radiomic features meeting a predetermined condition from the top predetermined number of radiomic features in the radiomic feature sequence to form a radiomic feature candidate set, wherein the predetermined condition is r>0.9 and p<0.05, wherein r is a correlation coefficient of a Spearman's rank correlation test, and p is a statistical test value; and
selecting a second predetermined number of radiomic features from the radiomic feature candidate set by using a Least Absolute Shrinkage and Selection Operator (LASSO) algorithm, to form a feature set.

13. The computer device according to claim 9, wherein the training a plurality of machine learning models based on the sample dataset, and selecting an optimal trained machine learning model as the glioma chemotherapy response prediction model comprises:
dividing the sample dataset into a training set and a test set;
training the plurality of machine learning models by using the training set, to obtain a plurality of trained machine learning models;
testing the plurality of trained machine learning models by using the test set to obtain a test result of each of the plurality of trained machine learning models, wherein the test result comprises one or more of an Area Under Curve (AUC), sensitivity, specificity, and accuracy; and
selecting one of the plurality of trained machine learning models that has an optimal test result as the glioma chemotherapy response prediction model.

14. The computer device according to claim 13, wherein the plurality of machine learning models comprise a random forest model, a support vector machine model, an extreme gradient boosting model, and a linear regression model.

15. The computer device according to claim 9, wherein the plurality of machine learning models comprise a random forest model, a support vector machine model, an extreme gradient boosting model, and a linear regression model.

16. The computer device according to claim 9, wherein the glioma chemotherapy response prediction model is:
R=0.5046+0.0203×M₁+0.0645×M₂+0.0063×M₃+0.0614×M₄+0.0017×M₅−0.0015×M₆−0.0619×M₇−0.0704×M₈−0.0459×M₉−0.0000×M₁₀+0.0284×M₁₁−0.0403×M₁₂;
where “R” is a score of chemotherapy response sensitivity, when “R>0.5”, a predicted result is determined as chemosensitive, and when “R≤0.5”, the predicted result is determined as chemoresistant; and M₁, M₂, M₃, M₄, M₅, M₆, M₇, M₈, M₉, M₁₀, M₁₁, and M₁₂ denote strength of an original neighborhood gray-tone difference matrix, a zone variance of a log-σ-5-0-mm-3D gray-level size zone matrix, a zone percentage of the log-σ-5-0-mm-3D gray-level size zone matrix, a joint average of a wavelet-LLH gray-level co-occurrence matrix, a contrast of a log-σ-5-0-mm-3D neighborhood gray-tone difference matrix, a gray-level variance of a wavelet-LLH gray-level size zone matrix, a run-length variance of a wavelet-LLH gray-level run-length matrix, a short run-length emphasis of the wavelet-LLH gray-level run-length matrix, run-length non-uniformity normalized of the wavelet-LLH gray-level run-length matrix, run-length non-uniformity of the wavelet-LLH gray-level run-length matrix, a run-length entropy of the wavelet-LLH gray-level run-length matrix, and long-run-length low-gray-level emphasis of the wavelet-LLH gray-level run-length matrix, respectively.

17. A non-transitory computer readable storage medium, having a computer program stored thereon, wherein the computer program, when executed by a processor, implements a method for constructing a glioma chemotherapy response prediction model, wherein the method comprises:
acquiring a longitudinal multi-time-point magnetic resonance imaging dataset, wherein the longitudinal multi-time-point magnetic resonance imaging dataset comprises a plurality of magnetic resonance imaging sequences, each of the plurality of magnetic resonance imaging sequences comprises magnetic resonance image groups of high-grade gliomas from a same patient at least at three time points; the three time points are located before surgery, before adjuvant chemotherapy, and after completion of adjuvant chemotherapy, respectively; and each of the magnetic resonance image groups comprises one or more of a T1-weighted image, a T2-weighted image, and a contrast-enhanced T1-weighted image;
calculating a volume change of residual tumors in a magnetic resonance image group before adjuvant chemotherapy and in a magnetic resonance image group after completion of adjuvant chemotherapy in each of the plurality of magnetic resonance imaging sequences as a volume change corresponding to each of the plurality of magnetic resonance imaging sequences;
determining chemotherapy sensitivity corresponding to each of the plurality of magnetic resonance imaging sequences according to the volume change corresponding to each of the plurality of magnetic resonance imaging sequences;
extracting and screening features from a magnetic resonance image group before surgery in each of the plurality of magnetic resonance imaging sequences to obtain a feature set corresponding to each of the plurality of magnetic resonance imaging sequences;
using the feature set corresponding to each of the plurality of magnetic resonance imaging sequences as an input and the chemotherapy sensitivity corresponding to each of the plurality of magnetic resonance imaging sequences as a label, to construct a sample dataset; and
training a plurality of machine learning models based on the sample dataset, and selecting an optimal trained machine learning model as the glioma chemotherapy response prediction model.

18. The non-transitory computer readable storage medium according to claim 17, wherein the calculating a volume change of residual tumors in a magnetic resonance image group before adjuvant chemotherapy and in a magnetic resonance image group after completion of adjuvant chemotherapy in each of the plurality of magnetic resonance imaging sequences comprises:
calculating a volume of a residual tumor in the magnetic resonance image group before adjuvant chemotherapy as a first volume by using the following formula:
V₁=N₁×V₀;
where V₁ is the volume of the residual tumor in the magnetic resonance image group before adjuvant chemotherapy, which is the first volume, N₁ is a number of voxels contained in a residual tumor area in the magnetic resonance image group before adjuvant chemotherapy, and V₀ is a volume of a single voxel;
calculating a volume of a residual tumor in the magnetic resonance image group after adjuvant chemotherapy as a second volume by using the following formula:
V₂=N₂×V₀;
where V₂ is the volume of the residual tumor in the magnetic resonance image group after completion of adjuvant chemotherapy, which is the second volume, and N₂ is a number of voxels contained in a residual tumor area in the magnetic resonance image group after completion of adjuvant chemotherapy; and
calculating the volume change according to the first volume and the second volume by using the following formula:
$$Δ V = \frac{{V}_{1} - {V}_{2}}{{V}_{1}} \times 100 \% ;$$
where “ΔV” is the volume change.

19. The non-transitory computer readable storage medium according to claim 18, wherein the determining chemotherapy sensitivity corresponding to each of the plurality of magnetic resonance imaging sequences according to the volume change corresponding to each of the plurality of magnetic resonance imaging sequences comprises:
determining the chemotherapy sensitivity as chemosensitive in response to determining that “ΔV” is greater than or equal to “65%”, and determining the chemotherapy sensitivity as chemoresistant in response to determining that “ΔV” is less than “65%”.

20. The non-transitory computer readable storage medium according to claim 18, wherein the extracting and screening features from a magnetic resonance image group before surgery in each of the plurality of magnetic resonance imaging sequences to obtain a feature set corresponding to each of the plurality of magnetic resonance imaging sequences comprises:
extracting features of the magnetic resonance image group before surgery in the magnetic resonance image sequence, to obtain radiomic features of the magnetic resonance image group before surgery in the magnetic resonance image sequence;
calculating distinctiveness of each radiomic feature by using a Maximum Relevancy Minimum Redundancy (mRMR) algorithm, and ranking each radiomic feature in a descending order of distinctiveness to obtain a radiomic feature sequence;
performing correlation analysis on a top predetermined number of radiomic features in the radiomic feature sequence, and selecting radiomic features meeting a predetermined condition from the top predetermined number of radiomic features in the radiomic feature sequence to form a radiomic feature candidate set, wherein the predetermined condition is r>0.9 and p<0.05, wherein r is a correlation coefficient of a Spearman's rank correlation test, and p is a statistical test value; and
selecting a second predetermined number of radiomic features from the radiomic feature candidate set by using a Least Absolute Shrinkage and Selection Operator (LASSO) algorithm, to form a feature set.
